SEQUENCE LISTING (35 sequences)

SEQ ID NO: 1 - DNA, 9432 bp, Homo sapiens
  ref <301>: AUTHORS: Kimura, S., Umeno, M., Skoda, R.C., Meyer, U.A. and Gonzalez, F.J.
  ref <302>: TITLE: The human debrisoquine 4-hydroxylase (CYP2D) locus: sequence and idetification of the polymorphic CYP2D6 gene, a related gene, and a pseudogene
  ref <303>: JOURNAL: American Journal of Human Genetics
  ref <304>: VOLUME: 45
  ref <305>: ISSUE: 6
  ref <306>: PAGES: 889-904
  ref <307>: DATE: 1989
  ref <308>: DATABASE ACCESSION NUMBER: M33388.1
  ref <309>: DATABASE ENTRY DATE: 2004-11-22
  ref <313>: RELEVANT RESIDUES IN SEQ ID NO: (1)..(9432)
  ref <308>: DATABASE ACCESSION NUMBER: M33388.1
  ref <309>: DATABASE ENTRY DATE: 2004-11-22
  ref <313>: RELEVANT RESIDUES IN SEQ ID NO: (1)..(9432)
gaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgagcgtggcgcatttcccagctggaatccggtgtcgaagtgggggcggggaccgcacctgtgctgtaagctcagtgtgggtggcgcggggcccgcggggtcttccctgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggctcagtcggcaggtgccagaacgttccctgggaaggccccatggaagcccaggactgagccaccaccctcagcctcgtcacctcaccacaggactggctacctctctgggccctcagggatgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccaccccgggcttctgtttccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccaccctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggccaggtcagctgtggtgaaaggtagggatgcagccagctcagggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacggcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccgccccactccaggggaagaggagtgccagcccttaccacctgagtgggcacagtgtagcatttattcattagcccccacactggcctgaccatctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggctgcagtgagccgagatcgcatcattgcactccagcctggtcaacaagagtgaaactgtcttaaaaaaaaaatctataattgatatctttagaaagataaaactttgcattcatgaaataagaataggagggtctaaaataaaaatgttcaaacacccaccaccactaattcttgacaaaaatatagtctgggtgccttagctcatgcctgtaatcccagcattttgggaggctaaggcaggaggattgtttgagcctaggaattc

SEQ ID NO: 2 - DNA, 19 bp, Artificial
  FORWARD PRIMER
actggctccaagcatggca

SEQ ID NO: 3 - DNA, 19 bp, Artificial
  REVERSE PRIMER
tcgccctgcagagactcct

SEQ ID NO: 4 - DNA, 20 bp, Artificial
  FORWARD PRIMER
cctcagcctcgtcacctcac

SEQ ID NO: 5 - DNA, 20 bp, Artificial
  REVERSE PRIMER
cacgtgcagggcacctagat

SEQ ID NO: 6 - DNA, 23 bp, Artificial
  FORWARD PRIMER
ggccaaggactctgtacctccta

SEQ ID NO: 7 - DNA, 21 bp, Artificial
  REVERSE PRIMER
cagtcctgtggtgaggtgacg

SEQ ID NO: 8 - DNA, 21 bp, Artificial
  FORWARD PRIMER
accgggcacctgtactcctca

SEQ ID NO: 9 - DNA, 22 bp, Artificial
  REVERSE PRIMER
gcatgagctaaggcacccagac

SEQ ID NO: 10 - DNA, 46 bp, Artificial
  ASPE PRIMER
tacaacatctcattaacatatacacctggacaacttggaagaaccc

SEQ ID NO: 11 - DNA, 48 bp, Artificial
  ASPE PRIMER
ttaaacaatctactattcaatcacagcctggacaacttggaagaaccg

SEQ ID NO: 12 - DNA, 44 bp, Artificial
  ASPE PRIMER
acatcaaattctttcaatatcttctgagctgctaactgagcaca

SEQ ID NO: 13 - DNA, 44 bp, Artificial
  ASPE PRIMER
taatttctttcatctctacataactgagctgctaactgagcacg

SEQ ID NO: 14 - DNA, 43 bp, Artificial
  ASPE PRIMER
aatcaacacacaataacattcatatggggcgaaaggggcgtcc

SEQ ID NO: 15 - DNA, 43 bp, Artificial
  ASPE PRIMER
aataacaactcactatatcataactggggcgaaaggggcgtct

SEQ ID NO: 16 - DNA, 43 bp, Artificial
  ASPE PRIMER
acaaatatctaactactatcacaacgctgggctgcacgctacc

SEQ ID NO: 17 - DNA, 43 bp, Artificial
  ASPE PRIMER
taaatcaatactcataatctcactcgctgggctgcacgctact

SEQ ID NO: 18 - DNA, 43 bp, Artificial
  ASPE PRIMER
atctcaattacaataacacacaaacggcctcctcggtcaccca

SEQ ID NO: 19 - DNA, 45 bp, Artificial
  ASPE PRIMER
tacacaatattcatcataactaacggcggcctcctcggtcacccc

SEQ ID NO: 20 - DNA, 45 bp, Artificial
  ASPE PRIMER
caatttacatttcactttcttatccctcctgctcatgatcctaca

SEQ ID NO: 21 - DNA, 45 bp, Artificial
  ASPE PRIMER
cttaaactctacttacttctaattcctcctgctcatgatcctacc

SEQ ID NO: 22 - DNA, 44 bp, Artificial
  ASPE PRIMER
tcatcactttctttactttacattcgccttcgccaaccactccg

SEQ ID NO: 23 - DNA, 44 bp, Artificial
  ASPE PRIMER
tcacttattacttcacattatctacgccttcgccaaccactcct

SEQ ID NO: 24 - DNA, 42 bp, Artificial
  ASPE PRIMER
atatactttacactttcaacaaacctgaccctccctctgcag

SEQ ID NO: 25 - DNA, 42 bp, Artificial
  ASPE PRIMER
tacttctttactacaatttacaacctgaccctccctctgcac

SEQ ID NO: 26 - DNA, 44 bp, Artificial
  ASPE PRIMER
tacttaaacatacaaacttactcatgcagcaggttgcccagccc

SEQ ID NO: 27 - DNA, 44 bp, Artificial
  ASPE PRIMER
ctatcatttatctctttctcaatttgcagcaggttgcccagcct

SEQ ID NO: 28 - DNA, 45 bp, Artificial
  ASPE PRIMER
atactttacaaacaaataacacacagcttcaatgatgagaacctg

SEQ ID NO: 29 - DNA, 44 bp, Artificial
  ASPE PRIMER
ctacaaacacttaactttatctttcttcaatgatgagaacctgt

SEQ ID NO: 30 - DNA, 42 bp, Artificial
  ASPE PRIMER
cttaacatttaacttctataacacggcagccactctcacctt

SEQ ID NO: 31 - DNA, 42 bp, Artificial
  ASPE PRIMER
cacaacaaattcttattcaattcaggcagccactctcacctc

SEQ ID NO: 32 - DNA, 42 bp, Artificial
  ASPE PRIMER
aactttctctctctattcttatttatggcgtttcatacttat

SEQ ID NO: 33 - DNA, 43 bp, Artificial
  ASPE PRIMER
cacttaattcattctaaatctatccgccagcacgttgacacct

SEQ ID NO: 34 - DNA, 46 bp, Artificial
  ASPE PRIMER
ttcaaacattcaaattactactacaccgcccgcctgtgcccatcac

SEQ ID NO: 35 - DNA, 44 bp, Artificial
  ASPE PRIMER
ttaatacaattctctctttctctacgcccgcctgtgcccatcat

(19) United States
(12) Patent Application Publication
(10) Pub. No.: US 2009/0215637 A1
(43) Pub. Date: Aug. 27, 2009

(54) Method of detecting mutations in the gene encoding cytochrome P450-2D6

(76) Inventors: Frank Merante, Etobicoke (CA); James D. Gordon, San Diego, CA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 11/631,197

(22) PCT Filed: Jun. 30, 2005
(86) PCT No.: PCT/CA05/01000

§ 371 Date: Oct. 23, 2008

Related U.S. Application Data
(60) Provisional application No. 60/583,605, filed on Jun. 30, 2004.

(51) Int. Cl. C40B 30/04; C12Q 1/68

(52) U.S. Cl. 506/9; 435/6

(57) ABSTRACT

The present invention describes a method for the simultaneous identification of two or more mutations located in the gene encoding Cytochrome P450-2D6. Multiplex detection is accomplished using multiplexed tagged allele specific primer extension (ASPE) and hybridization of such extended primers to a probe, preferably an addressable anti-tagged support.

### BACKGROUND OF THE INVENTION

1. Field of the Invention

The present invention relates to methods and kits for the detection of mutations located in the gene encoding Cytochrome P450-2D6.

2. Description of the Prior Art

Enzymes within the cytochrome P450 family are involved in the phase one metabolism of a wide range of small molecules including a large number of prescription drugs. CYP2D6 (Debrisoquine-4-hydroxylase) is involved in the metabolism of at least 58 clinically relevant drugs. Drug metabolism defects resulting from mutations in CYP2D6 can cause the accumulation of drugs to toxic levels thereby contributing to potential adverse drug reactions (ADRs).

The gene encoding cytochrome P450 2D6 is located on chromosome 22q13.1. Two spliced pseudogenes (CYP2D7P and CYP2D8P) that show a high degree of DNA sequence homology with CYP2D6 lie immediately upstream of CYP2D6 as shown in FIG. 1. The gene consists of nine exons arranged in a region of approximately 4.5 Kbp. The gene encodes the enzyme debrisoquine 4-hydroxylase. FIG. 1 shows the arrangement of the CYP2D6 gene and its relation to the two pseudogenes as well as a region deleted or duplicated in the major genomic variations.

A consensus nomenclature for Cytochrome P450 genes has been described by Daly et al. (1996). According to this nomenclature, alleles are denoted CYP plus the gene name (i.e. CYP2D6) followed by an asterisk and then the mutation as an Arabic numeral. For the most part the different CYP2D6 alleles are haplotypes consisting of a number of point mutations or small variants occurring together within the same gene. Subtypes of the same allele are further designated by letters (i.e. CYP2D6*4A). The mutations described for CYP2D6 are listed in the official homepage for the Human P450 Allele Nomenclature Committee (http://www.imm.ki.se/CYPalleles/default.htm). Numbering of nucleotide positions in the database is based on their position in the genomic entry (Genbank accession # M33388 or SEQ ID No: 1) but the numbering starts at the A in the ATG rather than using the number in the Genbank entry.

Genetic testing can be used to identify individuals at risk for ADRs based on their genetic profile and allow physicians to alter dosing regimens or choose alternate drugs to reduce the potential risk of an ADR. A need exists, however, for a rapid, and accurate test for the detection of specific mutations in the gene encoding CYP2D6.

Multiplex Allele Specific Primer Extension and Solid Support Detection of Mutations

Multiplex allele specific primer extension, and hybridization of extended primers to a solid support is described in the prior art. ASPE technology has been generally described in U.S. Pat. No. 4,851,331. The technology is designed to identify the presence or absence of specific polymorphic sites in the genome.

Multiplex ASPE in conjunction with hybridization to a support for mutation detection can be described generally as follows:

1) Amplifying regions of DNA comprising polymorphic loci utilizing a multiplexed, PCR.

2) Allele specific extension of primers wherein the amplified regions of DNA serve as target sequences for the allele specific extension. Extension primers that possess a 3′ terminal nucleotide which form a perfect match with the target sequence are extended to form extension products. Modified nucleotides are incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur.

3) Hybridizing the extension product to a probe on a solid support, such as a microarray, wherein the probe is complementary to the 5′ end of the extension product.

The extension primers used in a methodology as described above, possess unique sequence tags at their 5′ ends. For example, the sequence tags may allow the extension products to be captured on a solid support.

Variations of the above technology have been described, for example, in U.S. Pat. No. 6,287,778 and PCT Application (WO 00/47766).

It is an object of the present invention provide a cost effective, fast, and accurate method for identifying variants in the gene encoding CYP2D6.

### SUMMARY OF THE INVENTION

In one embodiment, the present invention provides a method for detecting the presence or absence of mutations in a sample selected from the group of mutations identified in Table 1, the method comprising the steps of:

Amplifying regions of DNA which may contain the above mentioned mutations using at least two PCR primer pairs selected from the group of PCR pairs consisting of SEQ ID NO.: 2 and SEQ ID NO: 3, SEQ ID NO.: 4 and SEQ ID NO: 5, SEQ ID NO.: 6 and SEQ ID NO: 7, and SEQ ID NO.: 8 and SEQ ID NO: 9.

Hybridizing at least two tagged allele specific extension primers, the allele specific extension primers selected from the group consisting of SEQ ID NO: 10 to SEQ ID NO: 35, to a complementary region of amplified DNA, each tagged allele specific primer having a 3′ portion complementary to a region of the amplified DNA, a 3′ terminal nucleotide complementary to one allele of one of the mutation sites (wild type or mutant) mentioned above, and a 5′ portion complementary to a probe sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to a probe and detection of labelled extension products. Detection of a labelled extension product is indicative of the presence of the allele complementary to the 3′-terminal nucleotide of the ASPE primer. In the absence of a labelled extension product, it is determined that the allele corresponding to the 3′ end of the ASPE primer is not present in the sample.

In another embodiment, the present invention provides a kit for use in detecting the presence or absence of at least two mutations identified in Table 1, the kit including at least two tagged allele specific extension primers selected from the group consisting of SEQ ID NO: 10 to SEQ ID NO: 35, and two per primer pairs selected from the group consisting of SEQ ID NO.: 2 and SEQ ID NO: 3, SEQ ID NO.: 4 and SEQ ID NO: 5, SEQ ID NO.: 6 and SEQ ID NO: 7, and SEQ ID NO.: 8 and SEQ ID NO: 9.

In another embodiment the present invention provides a method for detecting the presence or absence of nucleotide variants at polymorphic sites in the gene encoding cytochrome P450-2D6, said variants selected from the group of variants listed in table 1, the method comprising the steps of;

a) amplifying regions of DNA containing the variants to form amplified DNA products;

b) hybridizing at least two tagged allele specific extension primers to a complementary target sequence in the amplified DNA products, wherein each tagged allele specific extension primer has a 3′-end hybridizing portion capable of hybridizing to the amplified DNA, and wherein the 3′ end hybridizing portion of the at least two tagged allele specific extension primers comprise a sequence selected from the group consisting of bases 25 and up of SEQ ID NO: 10 to SEQ ID NO: 35, and a 5′-end tag portion complementary to a corresponding probe sequence, the terminal nucleotide of the 3′ end hybridizing portion being either complementary to a suspected variant nucleotide or to the corresponding wild type nucleotide of the site;

c) extending the at least two tagged allele specific extension primers, using labelled nucleotides, if the terminal nucleotide of the 3′ end hybridizing portion is a perfect match to an allele of one of the polymorphic sites in the amplified DNA products;

d) hybridizing the at least two tagged allele specific extension primers to the corresponding probe sequence and detecting the presence of labelled extension products.

In another embodiment, the present invention provides, a method for detecting the presence or absence of nucleotide variants at polymorphic sites in the gene encoding cytochrome P450-2D6, said variants selected from the group of variants listed in table 1, the method comprising the steps of;

a) amplifying regions of DNA containing the variants to form amplified DNA products;

b) hybridizing at least two tagged allele specific extension primers to a complementary target sequence in the amplified DNA products, wherein the at least two tagged allele-specific extension primers are selected from the group consisting of SEQ ID NO: 10 to SEQ ID NO: 35, each tagged allele specific extension primer having a 3′-end hybridizing portion capable of hybridizing to the amplified DNA, and a 5′-end tag portion complementary to a corresponding probe sequence, the terminal nucleotide of the 3′ end hybridizing portion being either complementary to a suspected variant nucleotide or to the corresponding wild type nucleotide of the site;

c) extending the at least two tagged allele specific extension primers, using labelled nucleotides, if the terminal nucleotide of the 3′ end hybridizing portion is a perfect match to an allele of one of the polymorphic sites in the amplified DNA products;

d) hybridizing the at least two tagged allele specific extension primers to the corresponding probe sequence and detecting the presence of labelled extension products.

In another embodiment, the present invention provides a kit for detecting the presence or absence of nucleotide variants at polymorphic sites in the gene encoding cytochrome P450-2D6, said variants selected from the group of variants listed in table 1, said kit comprising a set of at least two tagged allele specific extension primers wherein each tagged allele specific extension primer has a 3′-end hybridizing portion including a 3′ terminal nucleotide being either complementary to a suspected variant nucleotide or to the corresponding wild type nucleotide of one of the polymorphic sites and a 5′-end tag portion complementary to a corresponding probe sequence, and wherein the at least two tagged allele-specific extension primers are selected from the group consisting of SEQ ID NO: 10 to SEQ ID NO: 35.

In another embodiment the present invention provides A kit for detecting the presence or absence of nucleotide variants at polymorphic sites in the gene encoding cytochrome P450-2D6, said variants selected from the group of variants listed in table 1, said kit comprising a set of PCR amplification primers for amplifying regions of DNA containing the at least two polymorphic sites, said set comprising at least two pairs of PCR primers selected from the group of pairs consisting of:

### SEQ ID NO: 2 and SEQ ID NO: 3, SEQ ID NO: 4 and SEQ ID NO: 5, SEQ ID NO: 6 and SEQ ID NO: 7, and SEQ ID NO: 8 and SEQ ID NO: 9.

### BRIEF DESCRIPTION OF THE DRAWINGS

These and other features of the preferred embodiments of the invention will become more apparent in the following detailed description in which reference is made to the appended drawings wherein:

FIG. 1 depicts the arrangement of the CYP2D6 gene.

FIG. 2 depicts a general overview of steps of the present invention.

FIGS. 3 to 7 present genotyping results obtained using the method of the present invention.

### DESCRIPTION OF THE PREFERRED EMBODIMENTS

The following terms used in the present application will be understood to have the meanings defined below.

The term “mutations” as used herein refers to a number of classes of alteration in a nucleotide sequence including but not limited to the eight single base pair substitutions, two single base pair deletions, and one, three base pair deletion identified by the method of the present invention.

The terms “oligonucleotide” and “polynucleotide” as used in the present application refer to DNA sequences being of greater than one nucleotide in length. Such sequences may exist in either single or double-stranded form. Examples of oligonucleotides described herein include PCR primers, ASPE primers, and anti-tags.

The term “allele” is used herein to refer to variants of a nucleotide sequence.

The expression “allele specific primer extension (ASPE)”, as used herein, refers to a mutation detection method utilizing primers which hybridize to a corresponding DNA sequence and which are extended depending on the successful hybridization of the 3′ terminal nucleotide of such primer. Amplified regions of DNA serve as target sequences for ASPE primers. ASPE primers include a 3′ end-hybridizing portion which hybridizes to the amplified regions of DNA. ASPE primers that possess a 3′ terminal nucleotide which form a perfect match with the target sequence are extended to form extension products. Modified nucleotides can be incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur unless the polymerase used for extension inadvertently possesses exonuclease activity or is prone to misincorporation.

The term “genotype” refers to the genetic constitution of an organism More specifically, the term refers to the identity of alleles present in an individual. “Genotyping” of an individual or a DNA sample refers to identifying the nature, in terms of nucleotide base, of the two alleles possessed by an individual at a known polymorphic site.

The term “polymorphism”, as used herein, refers to the coexistence of more than one form of a gene or portion thereof.

The term “PCR”, as used herein, refers to the polymerase chain reaction. PCR is a method of amplifying a DNA base sequence using a heat stable polymerase and a pair of primers, one primer complementary to the (+)-strand at one end of the sequence to be amplified and the other primer complementary to the (−) strand at the other end of the sequence to be amplified. Newly synthesized DNA strands can subsequently serve as templates for the same primer sequences and successive rounds of heat denaturation, primer annealing and strand elongation results in rapid and highly specific amplification of the desired sequence. PCR can be used to detect the existence of a defined sequence in a DNA sample.

The term “primer”, as used herein, refers to a short single-stranded oligonucleotide capable of hybridizing to a complementary sequence in a DNA sample. A primer serves as an initiation point for template dependent DNA synthesis. Deoxyribonucleotides can be joined to a primer by a DNA polymerase. A “primer pair” or “primer set” refers to a set of primers including a 5′upstream primer that hybridizes with the complement of the 5′ end of the DNA sequence to be amplified and a 3′ downstream primer that hybridizes with the 3′ end of the DNA sequence to be amplified. The term “PCR primer” as used herein refers to a primer used for a PCR reaction. The term “ASPE primer” as used herein refers to a primer used for an ASPE reaction.

The term “tag” as used herein refers to an oligonucleotide sequence that is coupled to an ASPE primer. The sequence is generally unique and non-complementary to the human genome while being substantially complementary to a probe sequence. The probe sequence may be, for example, attached to a solid support. Tags serve to bind the ASPE primers to a probe.

The term “tagged ASPE primer” as used herein refers to an ASPE primer that is coupled to a tag.

The term “anti-tag” or “probe” as used herein refers to an oligonucleotide sequence having a sequence complementary to, and capable of hybridizing to, the tag sequence of an ASPE primer. The “anti-tag” may be coupled to a support.

The term “wild type” or “wt” as used herein refers to the normal, or non-mutated, or functional form of a gene.

The term “homozygous wild-type” as used herein refers to an individual possessing two copies of the same allele, such allele characterized as being the normal and functional form of a gene.

The term “heterozygous” or “HET” as used herein refers to an individual possessing two different alleles of the same gene.

The term “homozygous mutant” as used herein refers to an individual possessing two copies of the same allele, such allele characterized as the mutant form of a gene.

The term “mutant” as used herein refers to a mutated, or potentially non-functional form of a gene.

The term “deletion” as used herein refers to a mutation in which a portion of genomic DNA is deleted from a gene. The deletion may serve to eliminate all enzyme activity contributed by the chromosome where the deletion is located.

The term “duplication” as used herein refers to a mutation in which multiple copies of a gene may be present on an affected chromosome. The duplication may result in overproduction of an enzyme due to the presence of multiple copies of a specific gene.

The present invention was developed in response to a need for a rapid, highly specific, and cost-effective method to genotype individuals susceptible to adverse drug reactions. More specifically, the present invention provides a method for identifying individuals who may have drug metabolism defects resulting from mutations in the CYP2D6 gene.

The present invention provides a novel, multiplex method of detecting multiple mutations located in the gene encoding CYP2D6. Specifically, the methodology can be used for the detection of the presence or absence of two or more mutations selected from the group consisting of the mutations identified in Table 1. In a preferred embodiment, the present invention provides a method of detecting the presence or absence of all the mutations identified in Table 1.

TABLE 1
Mutations of the Gene Encoding CYP2D6

| | Major | | | PCR |
|---|---|---|---|---|
| Variant | Allele †- | | Effect | Amplimer |
| / | Gene | | ↑ Gene | α |
| | Duplication | | Copy | |
| / | *5 | | Gene | β |
| | | | Deletion | |
| −158C > G | *2A Promoter | | Expression | α |
| 100C > T | *4 | (A-L) | P34S | α |
| | *10 | (A, B) | | |
| 124G > A | *12 | | G42R | α |
| 883G > C | *11 | | Splicing | α |
| 1023C > T | *17 | | T1071 | α |
| 17077 > del | *6 | (A-D) | Frameshift | α |
| 1758G > T | *8 | | Stop | α |
| | | | Codon | |
| 1846G > A | *4 | (A-L) | Splicing | α |
| 2549A > del | *3 | (A, B) | Frameshift | β |
| 2613-2615 | *9 | | K281Δ | β |
| delAGA | | | | |

| | | | |
|---|---|---|---|
| 2850C > T | *2, *17 | R296C | β |
| 2935A > C | *7 | H324P | β |

↑ For *4, no *4I designation exists

The positive detection of one or more of the mutations identified in Table 1 may be indicative of an individual having a predisposition to adverse drug reactions.

FIG. 1 shows the location of the variants assayed for in the method of the present invention and the two coding region PCR amplimers used in the method (discussed further below).

The present invention is further characterized by a high level of specificity. Such specificity is required in order to ensure that any result generated is a true representation of the genomic target and not simply the result of non-specific interactions occurring between reagents present in reactions. This is especially important for multiplexed DNA-based tests where the numerous sequences present in the reaction mixture, most of which are non-complementary, may interact non-specifically depending on the reaction conditions.

The methodology of the present invention utilizes the combination of multiplex ASPE technology with hybridization of tagged and labelled extension products to probes in order to facilitate detection. Such methodology is suitable for high-throughput clinical genotyping applications.

In one embodiment, the present invention provides a method for detecting the presence or absence of mutations in a sample selected from the group of mutations identified in Table 1, the method comprising the steps of:

Amplifying regions of DNA which may contain the above mentioned mutations.

Hybridizing at least two tagged allele specific extension primers to a complementary region of amplified DNA, each tagged allele specific primer having a 3′ portion complementary to a region of the amplified DNA, a 3′ terminal nucleotide complementary to one allele of one of the mutation sites (wild type or mutant) mentioned above, and a 5′ portion complementary to a probe sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to aprobe and detection of labelled extension products. Detection of a labelled extension product is indicative of the presence of the allele complementary to the 3′-terminal nucleotide of the ASPE primer. In the absence of a labelled extension product, it is determined that the allele corresponding to the 3′ end of the ASPE primer is not present in the sample.

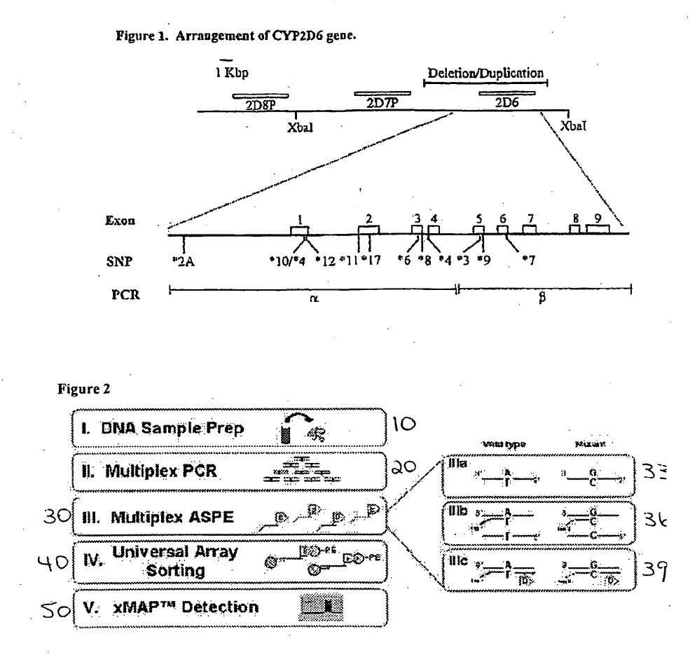

A general overview of one example of the above-mentioned method is presented in FIG. 2. A DNA sample is first prepared 10 using methods known in the art. Multiplex PCR amplification 20 is conducted in order amplify regions of DNA containing variant sites in the gene encoding cytochrome P450-2D6. A multiplex ASPE reaction 30 is then conducted. By example only, 33 illustrates a wild type and a mutant allele of a gene. At step 36 ASPE primers are hybridized to amplified regions of DNA. If the 3′ terminal nucleotide of an ASPE primer is complementary to a corresponding nucleotide in the target sequence, a labelled extension product is formed 39 as will be described further below. The ASPE may be sorted on an addressable universal sorting array 40 wherein the presence of a labelled extension product may be detected using, for example, XMAP detection 50.

FIG. 1 shows the location of the variants assayed for in the method of the present invention, and the two coding region PCR amplimers used in the assay.

In addition to the small nucleotide variations listed in Table 2, two large genomic rearrangements also affect CYP2D6 activity. The CYP2D6*5 allele is characterized by the deletion of 12.1 Kbp of genomic DNA including the entire 2D6 Gene. This deletion has a null phenotype eliminating all enzyme activity contributed by the chromosome on which it lies.

The CYP2D6××n allele is characterized by the tandem duplication of the 2D6 gene. The repeated unit is roughly 12.1 Kbp in length and is, in fact, identical to the region deleted in 2D6*5. In most cases the duplication results in the presence of two copies of the 2D6 gene on the affected chromosome although it has been observed that in rare cases up to 13 duplicated copies can be present. Both the original and duplicated copies of 2D6 are transcriptionally and translationally active resulting in overproduction of the enzyme and an ultra r phenotype (greater than normal levels of CYP2D6 activity). Most occurrences of the 2D6×n duplication have been observed with the 2D6*1 or 2D6*2 genotypes which are associated with the extensive metabolizer phenotype although the duplication has also been seen in association with 2D6*4 genotype.

### DNA Sample Preparation

Patient samples can be extracted with a variety of methods known in the art to provide nucleic acid (most preferably genomic DNA) for use in the following method

### Amplification

In a first step, at least one region of DNA from the gene encoding CYP2D6 is amplified. The at least one region amplified contains mutation sites listed in table 1.

In a preferred embodiment of the present invention, PCR amplification of regions containing mutation sites in the gene encoding CYP2D6 is initiated using PCR primer pairs selected from the group of primer pairs consisting of: SEQ ID NO.: 2 and SEQ ID NO.: 3, SEQ NO.: 4 and SEQ ID NO.: 5, SEQ ID NO.: 6 and SEQ ID NO.: 7, and SEQ ID NO.: 8 and SEQ ID NO.: 9.

The relationships of each pair of primers to the mutations listed in Table 1 and to the detection of the deletion and duplication regions described above is presented in Table 2.

TABLE 2
Primer Pairs Used to Amplify Regions
Containing CYP2D6 Mutations

| PCR Primer Pair | Mutations Contained in Amplimer |
|---|---|
| SEQ ID NO: 2 and 3 | All mutations (α-primers) |
| SEQ ID NO: 4 and 5 | Duplication Regions |
| SEQ ID NO: 6 and 7 | All mutations (β-primers) |
| SEQ ID NO: 8 and 9 | Deletion Regions |

An individual skilled in the art will recognize that alternate PCR primers could be used to amplify the target polymorphic regions, and deletion and duplication regions, however, in a preferred embodiment the primers listed in Table 2 are selected due to their minimal non-specific interaction with other sequences in the reaction mixture.

The presence of the deletion and duplication genomic rearrangements are detected by a PCR based mechanism. These approaches are adapted from the work of Steen et al., (1995) for the detection of 2D6*5 (deletion) and Løvlie et al., (1996) for the detection of the 2D6×n (duplication). The basis of these approaches is to utilize a pair of PCR primers that will yield a product only when the particular genomic rearrangement (deletion or duplication) is present. Since the recombination event in both the deletion and duplication occurs with a 2.8 Kbp. repeat region, the PCR amplimer must span this region. Thus, the deletion, when present, would generate a product of 3.5 Kb. While the duplication, when present, would generate a product of 3.2 Kb. The deletion and duplication primers cannot be used in the same PCR reaction since the mixing of primers would generate a signal from all WT genomic DNA samples. To minimize the number of PCR reactions performed the megaplex reactions are multiplexed by combining the α-primers with the duplication primers (the α reaction set) and the β-primers with the duplication primers (the β reaction set), as outlined in the example provided below.

### ASPE

The ASPE step of the method of the present invention is conducted using tagged ASPE primers selected from the group of ASPE primers consisting of SEQ ID NO: 10 to SEQ ID NO.: 35.

The ASPE primer set of the present invention has been optimized to ensure high specificity and accuracy of diagnostic tests utilizing such allele specific primers.

Table 3 presents a listing of the ASPE primers used in a preferred embodiment of the present invention. The suffix “wt” indicates an ASPE primer used to detect the wild type form of the gene encoding CYP2D6 at a specific mutation site. The suffix “mut” indicates an ASPE primer used to detect a mutant form of the gene encoding CYP2D6 at a specific mutation site. The suffix “dup” indicates an ASPE primer used to detect a duplication region. The suffix “del” indicates an ASPE primer used to detect a deleted region. Bases 1 to 24 of each of SEQ ID NO.: 10 to SEQ ID NO: 35 are the 5′ portions of the ASPE primers that are complementary to specific probe sequences. Although the specific sequences listed in table 3 are preferred, in alternate embodiments of the present invention, it is possible to combine different 5′ portions of the sequences in Table 3 (bases 1 to 24 of SEQ ID NOs: 10 to 35) with different 3′ end hybridizing portions of the sequences in Table 3 (bases 25 and up of SEQ ID NOs: 10 to 35).

The orientation of each of the ASPE primers is also presented in Table 3.

TABLE 4
P450-2D6 ASPE Primer Sequences

| SEQ ID | | |
|---|---|---|
| NO: | Site Detected | Direction |
| 10 | SNP17promo-wt | Forward |
| 11 | L-SNP17promo-mut | Forward |
| 12 | SNP13*3wt | Forward |
| 13 | SNP13*3mut | Forward |
| 14 | SNP12*4wt | Reverse |
| 15 | SNP12*4mut | Reverse |
| 16 | SNP1*10wt | Forward |
| 17 | SNP1*10mut | Forward |
| 18 | SNP10*6wt | Forward |
| 19 | L-SNP10*6mut | Forward |
| 20 | SNP15*7wt | Forward |
| 21 | SNP15*7mut | Forward |
| 22 | SNP11*8wt | Forward |
| 23 | SNP11*8mut | Forward |
| 24 | SNP3*11wt | Forward |
| 25 | SNP3*11mut | Forward |
| 26 | SNP2*12wt | Reverse |
| 27 | SNP2*12mut | Reverse |
| 28 | SNP14*17wt | Forward |
| 29 | S2-SNP14*17 | Forward |
| 30 | S-SNP20*9wt | Reverse |
| 31 | S-SNP20*9mut | Reverse |
| 32 | Sdup | Forward |
| 33 | S*5del | Forward |
| 34 | L1*17(1023wt) | Forward |
| 35 | S1*17(123mut) | Forward |

The 3′ end hybridizing portion of the extension primer is hybridized to the amplified material. Where the 3′ terminal nucleotide of the 3′ end hybridizing portion of the ASPE primer is complementary to the polymorphic site, primer extension is carried out using modified nucleotides. Where the 3′ terminal nucleotide of the ASPE primer is not complementary to the polymorphic region, no primer extension occurs.

In one embodiment, labelling of the extension products is accomplished through the incorporation of biotinylated nucleotides into the extension product which may be identified using fluorescent (Streptavidin-Phycoerythrin) or chemiluminescent (Streptavidin-Horseradish Peroxidase) reactions. However, an individual skilled in the art will recognize that other belling techniques may be utilized. Examples of labels useful for detection include but are not limited to radiolabels, fluorescent labels (e.g fluorescein and rhodamine), nuclear magnetic resonance active labels, positron emitting isotopes detectable by a positron emission tomography (“PET”) scanner, and chemiluminescers such as luciferin, and enzymatic markers such as peroxidase or phosphatase.

Each ASPE primer used in the methodology as described above, possess a unique sequence tag at their 5′ ends. The sequence tags allow extension products to be detected with a high degree of specificity, for example, through capture on a solid support in order to facilitate detection.

Detection

The tagged 5′ portions of the allele specific primers of the present invention are complementary to probe sequences. Upon hybridization of the allele specific primers to a corresponding probe sequence the presence of extension products can be detected.

In a preferred embodiment, probes used in the methodology of the present invention are coupled to a solid support, for example a ‘universal’ bead-based microarray.

Examples of supports that can be used in the present invention include, but are not limited to, bead based microarrays and 2D glass microarrays. The preparation, use, and analysis of microarrays are well known to persons skilled in the art. (See, for example, Brennan, T. M. et al. (1995) U.S. Pat. No. 5,474,796; Schena, et al. (1996) Proc. Natl. Acad. Sci. 93:10614-10619; Baldescbweiler et al. (1995), PCT Application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662). Detection can be achieved through arrays using, for example, chemiluminescence or fluorescence technology for identifying the presence or absence of specific mutations.

Universal arrays function as sorting tools indirectly detecting the target of interest and are designed to be isothermal and minimally cross-hybridizing as a set. Examples of microarrays which can be used in the present invention include, but should not be limited to, Luminex's® bead based microarray systems, and Metrigenix's™ Flow Thru chip technology.

In one embodiment, for example, Luminex's 100 xMAP™ fluorescence based solid support microarray system is utilized. Anti-tag sequences complementary to the tag regions of the ASPE primers/extension products, described above, are coupled to the surface of internally fluorochrome-color-coded microspheres. An array of anti-tag microspheres is produced, each set of microspheres having its own characteristic spectral address. The mixture of tagged, extended, biotinylated ASPE primers is combined with the array of anti tagged microspheres and is allowed to hybridize under stringent conditions.

In a reaction mixture, a fluorescent reporter molecule (e.g. streptavidin-phycoerythrin) is used to detect labelled extension products which are synthesized when the terminal nucleotide of an ASPE primer is complementary to a corresponding nucleotide in the target sequence.

The reaction mixture, comprising microspheres, extension products etc. is injected into a reading instrument, for example Luminex's 100 xMAP™, which uses microfluidics to align the microspheres in single file. Lasers are used to illuminate the fluorophores both internal to the microspheres, and attached to the surface in the form of extension products hybridized to anti-tag sequences. The Luminex 100 xMAP™, interprets the signal received and identifies the presence of wild type and/or mutant alleles. The presence of the mutant allele of any one or more of the mutations presented in Table 2 may be indicative a predisposition for adverse drug reactions. Software can be provided which is designed to analyze data associated with the specific extension products and anti-tagged microspheres of the present invention.

In another embodiment, the Metrigenix Flow-Thru three dimensional microchannel biochip (Cheek, B. J., Steel A. B., Torres, M. P., Yu, Y., and Yang H. Anal. Chem. 2001, 73, 5777-5783) is utilized for genotyping as known in the art. In this embodiment, each set of microchannels represents a different universal anti-tag population Anti-tag sequences corresponding to the tag regions of the ASPE primers/extension products, described above, are attached to the inner surface of multiple microchannels comprising a cell. Multiple cells make up a chip. The reaction mixture, including biotinylated extension products flows through the cells in the presence of a chemiluminescent reporter substrate such as streptavidin-horseradish peroxidase. Microarray chips can be imaged using technology known in the art, such as an ORCA-ER CCD (Hamamatsu Photonics K. K., Hamamatsu City, Japan), and imaging software, in order to identify the genotype of an individual.

### Kits

In an additional embodiment, the present invention provides kits for the multiplex detection of mutations in the gene encoding CYP2D6.

A kit that can be used for detection of the mutations of interest may contain the following components including: a PCR primer mix for amplifying regions containing mutation sites of interest (optionally including dNTPs), an ASPE primer mix for generation of labelled extension products (optionally including dNTPs) and a solid support, such as microarray beads, the beads having anti-tags complementary to the tagged regions of the ASPE primers. In addition, an individual skilled in the art would recognize other components which could be included in such kits including, for example, buffers and polymerases.

Kits of the present invention may include PCR primer pairs, ASPE primers, and tagged supports for all the mutations to be detected, or may be customized to best suit the needs of an individual end user. For example, if an end user wishes to detect only 5 of the mutations in the CYP2D6 gene, a kit can be customized to include only the PCR primer pairs, ASPE primers, and support required for the detection of the desired mutations. As such, the end user of the product can design a kit to match their specific requirements. In addition, the end user can also control the tests to be conducted at the software level when using, for example, a universal bead based-microarray for detection. For example, software can be provided with a kit, such software reading only the beads for the desired mutations or by reporting only the results from the desired mutation data. Similar control of data reporting by software can be obtained when the assay is performed on alternate platforms.

An individual skilled in the art will recognize that although the present method has been described in relation to the specific mutations identified in Table 1, PCR primers and ASPE primers used to detect additional mutations could be included in the above method and kits.

### Example #1

### ASPE/Microarray Detection of Mutations in the Gene Encoding CYP2D6

The following represents an example protocol for use in the method of the present invention.

For each genomic sample being tested, two separate PCR reactions are performed. Each PCR reaction requires 25 ng genomic DNA (ie. 50 ng genomic DNA per sample). The first PCR (PCR-α) produces an alpha fragment (3.8 kb) (from PCR primer pair comprising SEQ ID NO: 2 and SEQ ID NO: 3) used to detect the variants shown in Table 1, as well as a duplication amplimer (3.2 kb) (from PCR primer pair comprising SEQ ID NO: 4 and SEQ ID NO: 5) which indicates the presence of the duplication genotype, if present. The second PCR (PCR-β) produces a beta fragment (2.6 kb)) (from PCR primer pair comprising SEQ ID NO: 6 and SEQ ID NO: 7) used to detect the variants shown in Table 2, as well as a deletion amplimer (3.5 kb) (from PCR primer pair comprising SEQ ID NO: 8 and SEQ ID NO: 9) indicative of the deletion genotype, if present. Following PCR amplification, the two reactions (PCR-α and PCR-β) are pooled. To enable efficient incorporation of biotin-dCTP during the Allele Specific Primer Extension (ASPE) reaction, the pooled PCR product is treated with Shrimp Alkaline Phosphatase (SAP) to inactivate any remaining nucleotides (particularly dCTP), and with Exonuclease 1 (EXO) to degrade any primers left over from the PCR reaction ASPE is then carried out using 26 universally-tagged primers (SEQ ID NO: 10 to SEQ ID NO: 35) supplied in the ASPE primer mix A 5 uL aliquot of the ASPE reaction is hybridized with the universal array (Bead Mix) in the presence of the hybridization buffer and incubated with Streptavidin, R-Phycoerythrin conjugate (reporter solution). Samples are read on the Luminex® 100 xMAP™ Instrument and signal is generated for each of the 12 small nucleotide variants as well as for the duplication and deletion amplimers (if present). These fluorescence values are then analyzed to determine whether the wild-type/mutant allele for each of the 12 small nucleotide variants has been detected or whether the samples carry an allele(s) with the deletion or duplication.

### Example #2

### Detection of Mutations in the Gene Encoding CYP2D6

1) Oligonucleotides

All oligonucleotides were synthesized by Integrated DNA Technologies (Coralville, Iowa). PCR primers were unmodified and were purified by standard desalting procedures. Universal anti-tags (probes) were 3′-C7 amino-modified for coupling to carboxylated microspheres. All anti-tags were reverse phase HPLC-purified. Chimeric ASPE primers which consisted of a 24mer universal tag sequence 5′ to the allele-specific sequence were also unmodified but were purified by polyacrylamide gel electrophoresis. Following reconstitution, exact oligo concentrations were determined spectrophotometrically using extinction coefficients provided by the supplier. Reconstituted oligos were scanned between 200 and 500 nm and absorbance was measured at 260 nm to calculate oligo concentration.

2) Reagents

Expand Long Template PCR System was purchased from Roche Diagnostics (Indianapolis Ind.). Platinum Tsp DNA Polymerase, individual dNTPs and biotin-dCTP were purchased from Invitrogen Corporation (Carlsbad, Calif.). Shrimp alkaline phosphatase and Exonuclease I were purchased from USB Corporation (Cleveland, Ohio). Carboxylated fluorescent microspheres were provided by Luminex Corporation (Austin, Tex.). The EDC cross-linker (1-ethyl-3-(3-dimethylaminopropyl) carbodiimide hydrochloride) was purchased from Pierce (Rockford, Ill.). OmniPur reagents including MES (2-(N-morpholino)ethane sulfonic acid), 10% SDS, NaCl, Tris, Triton X-100, Tween-20 and TE buffer were purchased from EM Science (Darmstadt, Germany). The streptavidin-conjugated phycoerythrin was obtained from Molecular Probes Inc. (Eugene, Oreg.).

3) Genotyping

a) MULTIPLEX PCR (two 2-plexes): Each PCR was carried out using 25 ng genomic DNA in a 10 μL final volume. A ‘no target’ PCR negative control was included with each assay run. The reaction consisted of 1× Expand Long Buffer 2,200 umol/L each dNTP, 0.75 units of Expand Long Enzyme Mix (Roche), with primers ranging from 0.45 to 1 umol/L. Samples were cycled in an MJ Research PTC-200 thermocycler (Waterdown Mass.) with cycling parameters set at 95° C. for 3 minutes followed by 35 cycles at 95° C. for 60 seconds, 66° C. for 30 seconds and 72° C. for 3 minutes. Samples were then held at 72° C. for 5 minutes and kept at 4° C. until use. Following completion of the PCR, the A and B reactions were pooled.

b) ALLELE-SPECIFIC PRIMER EXTENSION: Prior to the ASPE reaction, each pooled PCR reaction mixture was treated with shrimp alkaline phosphatase (SAP) to inactivate any remaining nucleotides (particularly dCTP) so that biotin-dCTP could be efficiently incorporated during the primer extension reaction. Each PCR reaction was also treated with exonuclease I (EXO) to degrade remaining PCR primers in order to avoid any interference with the tagged ASPE primers and the extension reaction itself. To each pooled sample (20 μL, 2 μL SAP (=2 units) and 0.5 μL EXO (=5 units) were added directly to the sample. Samples were then incubated at 37° C. for 30 minutes followed by a 15 minute incubation at 99° C. to inactivate the enzymes. Samples were then added directly to the ASPE reaction.

Multiplex ASPE was carried out using 5 uL of treated PCR product in a final volume of 20 uL. Each reaction consisted of 20 mmol/L Tris-HCl pH 8.4, 50 mmol/L KCl, 1.25 mmol/L MgCl2, 5 umol/L biotin-dCTP, 5 umol/L each of dATP, dGTP and dTFP, 1.5 units Platinum Tsp DNA Polymerase and 2.5 nmol/L ASPE primer pool. The ASPE reactions were incubated at 96° C. for 2 minutes and then subjected to 40 cycles at 94° C. for 30 seconds, 54° C. for 30 seconds and 74° C. for 60 seconds. Reactions were then held at 4° C. until use.

c) BEAD COUPLING: Amino-modified anti-tag sequences were coupled to carboxylated microspheres following Luminex's one-step carbodiimide coupling procedure. Briefly, 5×10⁶ microspheres were combined with 1 nmol NH₂-oligo in a final volume of 50 μL 0.1 mol/L MES, pH 4.5. A 10 mg/mL EDC working solution was prepared just prior to use and 2.5 μL was added to the bead mixture and incubated for 30 minutes. A second 2.5 μL aliquot of freshly prepared EDC was added followed by an additional 30 minute incubation. Following washes in 0.02% (v/v) Tween-20 and 0.1% (w/v) SDS, the anti-tag coupled beads were resuspended in 100 uL TE buffer (10 mmol/L Tris, pH 8.0, 1 mmol/L EDTA). Bead concentrations were determined using a Beckman Coulter Z2 Particle Count and Size Analyzer (Coulter Corp, Miami Fla.).

d) UNIVERSAL ARRAY HYBRIDIZATION: Each hybridization reaction was carried out using approximately 2500 beads of each of the 26 anti-tag bearing bead populations. The beads were combined in hybridization buffer (0.22 mol/L NaCl, 0.11 mol/L Tris, pH 8.0 and 0.088% (v/v) Triton X-100) and 45 μL of the mix were added to each well of an MJ Research 96-well plate (Waterdown Mass.). A 5 μL aliquot of each ASPE reaction was then added directly to each well. The samples were then heated to 96° C. for 1 minutes in an MJ Research PTC-200 followed by a one hour incubation at 37° C. Following this incubation, samples were filtered through a 1.2 um Durapore Membrane (Millipore Corp, Bedford, Mass.) and washed once using wash buffer (0.2 mol/L NaCl, 0.1 mol/L Tris, pH 8.0 and 0.08% (v/v) Triton X-100). The beads were then resuspended in 150 uL reporter solution (1 ug/mL streptavidin-conjugated phycoerythrin in wash buffer) and incubated for 15 minutes at room temperature. The reactions were read on the Luminex xMAP. Acquisition parameters were set to measure 100 events per bead population and a 100 uL sample volume. A gate setting was established prior to running the samples and maintained throughout the course of the study.

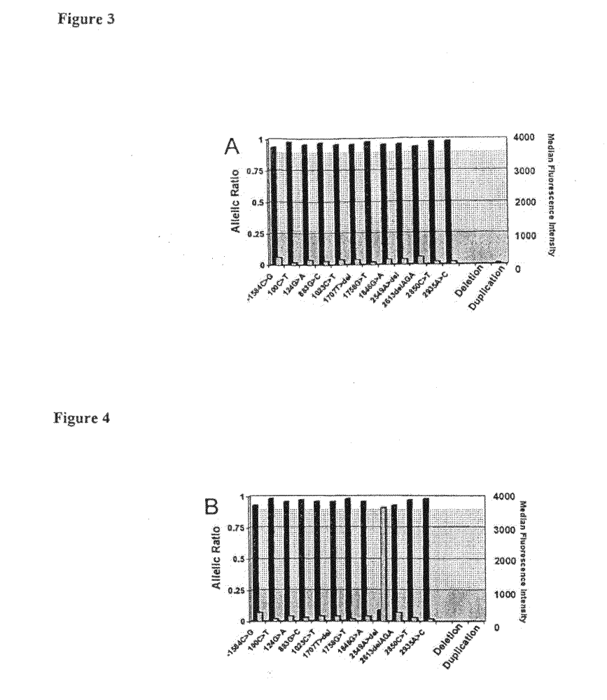

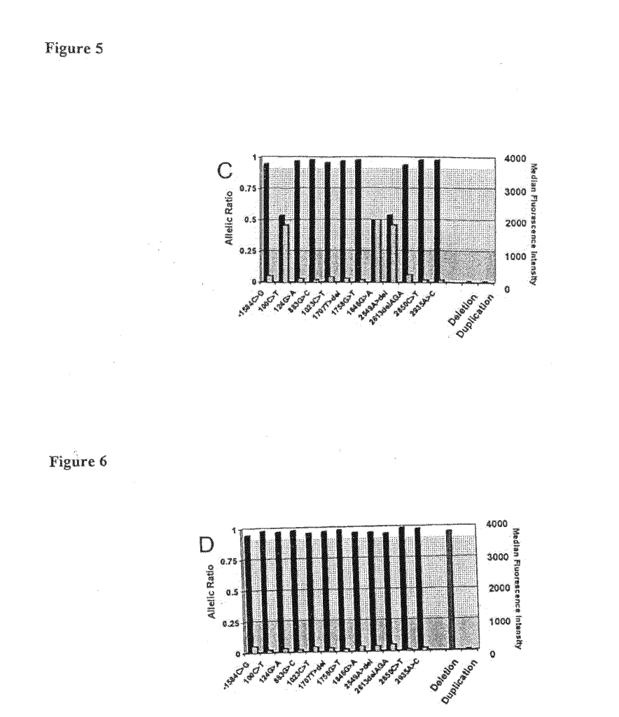

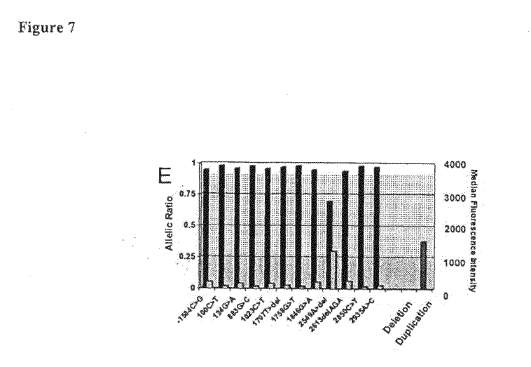

Representative results obtained with the kit of the present invention are presented in FIGS. 3 to 7. WT (dark bars) and mutant (light bars) allelic ratios are shown for small nucleotide variations while median fluorescent intensity is shown for the deletion and duplication. FIG. 3 shows results obtained for an individual who is WT for all alleles tested. FIG. 4 shows the results obtained from an individual mutant for the 2549A>del (*3) variant. FIG. 5 shows the result from an individual heterozygous for three variants; the 100C>T and 1846G>A variations are both found in the *4 allele. FIGS. 6 and 7 show the profiles seen with individuals with the gene deletion or duplication respectively.

All publications, patents and patent applications are herein incorporated by reference in their entirety to the same extent as if each individual publication, patent or patent application was specifically and individually indicated to be incorporated by reference in its entirety

Although the invention has been described with reference to certain specific embodiments, various modifications thereof will be apparent to those skilled in the art without departing from the spirit and scope of the invention as outlined in the claims appended hereto.

The embodiments of the invention in which an exclusive property or privilege is claimed are defined as follows:

1. A method for detecting the presence or absence of nucleotide variants at polymorphic sites in the gene encoding cytochrome P450-2D6, said variants selected from the group of variants listed in table 1, the method comprising the steps of;
a) amplifying regions of DNA containing the variants to form amplified DNA products;
b) hybridizing at least two tagged allele specific extension primers to a complementary target sequence in the amplified DNA products, wherein each tagged allele specific extension primer has a 3′-end hybridizing portion capable of hybridizing to the amplified DNA, and wherein the 3′ end hybridizing portion of the at least two tagged allele specific extension primers comprise a sequence selected from the group consisting of bases 25 and up of SEQ ID NO: 10 to SEQ ID NO: 35, and a 5′-end tag portion complementary to a corresponding probe sequence, the terminal nucleotide of the 3′ end hybridizing portion being either complementary to a suspected variant nucleotide or to the corresponding wild type nucleotide of the site;
c) extending the at least two tagged allele specific extension primers, using labelled nucleotides, if the terminal nucleotide of the 3′ end hybridizing portion is a perfect match to an allele of one of the polymorphic sites in the amplified DNA products;
d) hybridizing the at least two tagged allele specific extension primers to the corresponding probe sequence and detecting the presence of labelled extension products.

2. The method of claim 2 wherein the 5′-end tag portions of the at least two tagged allele specific primers comprises a sequence selected from the group consisting of bases 1 to 24 of SEQ ID NO: 10 to SEQ ID NO: 35.

3. The method of claim 1 wherein the probe sequence is coupled to a solid support.

4. The method of claim 3 wherein the solid support is selected from the group consisting of beads, spectrally coded beads, and a chip based microarray.

5. The method of claim 1 wherein the step of amplifying is conducted by PCR using a set of PCR amplification primers, said set comprising at least two pairs of PCR primers selected from the group of pairs consisting of:
SEQ ID NO: 2 and SEQ ID NO: 3, SEQ ID NO: 4 and SEQ ID NO: 5, SEQ ID NO: 6 and SEQ ID NO: 7, and SEQ ID NO: 8 and SEQ ID NO: 9.

6. A method for detecting the presence or absence of nucleotide variants at polymorphic sites in the gene encoding cytochrome P450-2D6, said variants selected from the group of variants listed in table 1, the method comprising the steps of;
a) amplifying regions of DNA containing the variants to form amplified DNA products;
b) hybridizing at least two tagged allele specific extension primers to a complementary target sequence in the amplified DNA products, wherein the at least two tagged allele-specific extension primers are selected from the group consisting of SEQ ID NO: 10 to SEQ ID NO: 35, each tagged allele specific extension primer having a 3′-end hybridizing portion capable of hybridizing to the amplified DNA, and a 5′-end tag portion complementary to a corresponding probe sequence, the terminal nucleotide of the 3′ end hybridizing portion being either complementary to a suspected variant nucleotide or to the corresponding wild type nucleotide of the site;
c) extending the at least two tagged allele specific extension primers, using labelled nucleotides, if the terminal nucleotide of the 3′ end hybridizing portion is a perfect match to an allele of one of the polymorphic sites in the amplified DNA products;
d) hybridizing the at least two tagged allele specific extension primers to the corresponding probe sequence and detecting the presence of labelled extension products.

7. The method of claim 6 wherein the probe sequence is coupled to a solid support.

8. The method of claim 7 wherein the solid support is selected from the group consisting of beads, spectrally coded beads, and a chip based microarray.

9. The method of claim 6 wherein the step of amplifying is conducted by PCR using a set of PCR amplification primers, said set comprising at least two pairs of PCR primers selected from the group of pairs consisting of:
SEQ ID NO: 2 and SEQ ID NO: 3, SEQ ID NO:4 and SEQ ID NO: 5, SEQ ID NO:6 and SEQ ID NO: 7, and SEQ ID NO: 8 and SEQ ID NO: 9.

10. A kit for detecting the presence or absence of nucleotide variants at polymorphic sites in the gene encoding cytochrome P450-2D6, said variants selected from the group of variants listed in table 1, said kit comprising a set of at least two tagged allele specific extension primers wherein each tagged allele specific extension primer has a 3′-end hybridizing portion including a 3′ terminal nucleotide being either complementary to a suspected variant nucleotide or to the corresponding wild type nucleotide of one of the polymorphic sites and a 5′-end tag portion complementary to a corresponding probe sequence, and wherein the at least two tagged allele-specific extension primers are selected from the group consisting of SEQ ID NO: 10 to SEQ ID NO: 35.

11. The kit of claim 10 further comprising a set of PCR amplification primers for amplifying regions of DNA containing the polymorphic sites, said set comprising at least two pairs of PCR primers selected from the group of pairs consisting of SEQ ID NO: 2 and SEQ ID NO: 3, SEQ ID NO: 4 and SEQ ID NO: 5, SEQ ID NO: 6 and SEQ ID NO: 7, and SEQ ID NO: 8 and SEQ ID NO: 9.

12. The kit of claim 10 further comprising a set of probes.

13. The kit of claim 12 wherein the set of probes are coupled to a support.

14. A kit for detecting the presence or absence of nucleotide variants at polymorphic sites in the gene encoding cytochrome P450-2D6, said variants selected from the group of variants listed in table 1, said kit comprising a set of PCR amplification primers for amplifying regions of DNA containing the at least two polymorphic sites, said set comprising at least two pairs of PCR primers selected from the group of pairs consisting of:
SEQ ID NO: 2 and SEQ ID NO: 3, SEQ ID NO: 4 and SEQ ID NO: 5, SEQ ID NO: 6 and SEQ ID NO: 7, and SEQ ID NO: 8 and SEQ ID NO: 9.

15. The kit of claim 14 further comprising a set of at least two tagged allele specific extension primers wherein each tagged allele specific extension primer has a 3′-end hybridizing portion capable of hybridizing to the amplified DNA, a 5′-end tag portion complementary to a corresponding probe sequence, the terminal nucleotide of the 3′ end hybridizing portion being either complementary to a suspected variant nucleotide or to the corresponding wild type nucleotide of the polymorphic sites.
